(19) United States
(12) Patent Application Publication
(10) Pub. No.: US 2006/0069355 A1
(43) Pub. Date: Mar. 30, 2006

(54) Medication dispensing apparatus with squeezable actuator

(75) Inventors: Jared Alden Judson, Topsfield, MA (US); Kenneth Alan Ritsher, Chicago, IL (US)

Correspondence Address: ELI LILLY & COMPANY, PATENT DIVISION, P.O. BOX 6288, INDIANAPOLIS, IN, 46206-6288, US

(73) Assignee: Jared Alden JUDSON and Kenneth Alan RITSHER

(21) Appl. No.: 10/531,505

(22) PCT Filed: Oct. 8, 2003
(86) PCT No.: PCT/US03/28565

§ 371 Date: Apr. 13, 2005

Related U.S. Application Data
(60) Provisional application No. 60/419,059, filed on Oct. 15, 2002.

(51) Int. Cl. A61M 5/00

(52) U.S. Cl. 604/211; 604/232

(57) ABSTRACT

A medication dispensing apparatus having an actuator that is laterally squeezable to dispense medication. The apparatus includes a supply of medication that is dispensed when a piston is advanced by a drive member. A dose to be dispensed is selectively set by operation of a dosing member, which operation causes the actuator to shift in a direction transverse to the direction the piston is advanced to dispense medication. The actuator shifts in this traverse direction from a ready position a distance that is a function of the selectively set dose. When the actuator is squeezed back to its ready position, the drive member is shifted to advance the piston to dispense the medication.

### BACKGROUND OF THE INVENTION

The present invention pertains to medication dispensing devices, and, in particular, to a portable medication dispensing device such as an injection pen.

Patients suffering from a number of different diseases frequently must inject themselves with medication. To allow a person to conveniently and accurately self-administer medicine, a variety of devices broadly known as injector pens or injection pens have been developed. Generally, these pens are equipped with a cartridge including a piston and containing a multi-dose quantity of liquid medication. A drive member, extending from within a base of the injection pen and operably connected with typically more rearward mechanisms of the pen that control drive member motion, is movable forward to advance the piston in the cartridge in such a manner to dispense the contained medication from an outlet at the opposite cartridge end, typically through a needle that penetrates a stopper at that opposite end. In disposable pens, after a pen has been utilized to exhaust the supply of medication within the cartridge, the entire pen is discarded by a user, who then begins using a new replacement pen. In reusable pens, after a pen has been utilized to exhaust the supply of medication within the cartridge, the pen is disassembled to allow replacement of the spent cartridge with a fresh cartridge, and then the pen is reassembled for it subsequent use.

One shortcoming of some injection pens is that operating the pen to dispense the medication is physically difficult for some users. For example, many types of injection pens have an injection button on a pen end opposite a needled end, and to inject medication this button is designed to be plunged with a digit, for example thumb, of the hand that grasps the pen. For some users, such as those who have limited hand strength, possibly due to the ailment being treated, providing sufficient force to so plunge the injection button may be problematic.

One injection pen disclosed in U.S. Pat. No. 5,584,815 utilizes an actuator that is laterally squeezed, rather than axially plunged, to dispense medication. While useful, and perhaps more readily operated than some other devices, this pen is not without its shortcomings. For one thing, the pen has a design that is too complicated and potentially expensive for some applications. Moreover, the complicated design results in a larger size that may make transporting and using the pen less convenient than desired.

Thus, it would be desirable to provide an apparatus that can overcome one or more of these and other shortcomings of the prior art.

### BRIEF SUMMARY OF THE INVENTION

In one form thereof, the present invention provides a medication dispensing apparatus including a housing, a drive member movable within the housing, a fluid container defining a medicine-filled reservoir with a movable piston at one end and an outlet at the other end, the piston engagable by the drive member to be advanced toward the outlet in a distal direction when the drive member is moved, and a dosing and injecting assembly for selectively shifting the drive member. The dosing and injecting assembly includes an actuator and a dosing member, the dosing member being operable to selectively set a dose to be delivered, and the actuator being movable relative to the housing in a direction transverse to the distal direction between a ready position and any one of a plurality of dosed positions. The actuator, responsive to the dosing member being operated to selectively set a particular dose to be delivered, moves from the ready position to a particular dosed position that corresponds to the particular dose selectively set. The drive member, responsive to the actuator being manually shifted from the particular dosed position back to the ready position, moves to shift the piston distally to cause the set dose to be delivered through the outlet.

One advantage of the present invention is that a medication dispensing apparatus can be provided which is simple and intuitive to operate, compact, and mechanically efficient.

Another advantage of the present invention is that a medication dispensing apparatus can be provided with a squeezable actuator that facilitates dispensing of medication.

Still another advantage of the present invention is that a medication dispensing apparatus can be provided with an actuator that laterally shifts away from the apparatus housing during dose selection in an amount directly proportional to the amount of the dose to be dispensed by operation.

Still another advantage of the present invention is that a medication dispensing apparatus can be provided having increased stability of its needle during injection due to the manner in which the apparatus is operated to force medicine through that needle.

Yet another advantage of the present invention is that a medication dispensing apparatus can be provided having an easy to read analog dial indicator.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a front view of a first embodiment of a medication dispensing apparatus with squeezable actuator of the present invention, which apparatus is arranged in a ready or ready-to-be-dosed state;

FIG. 2 is a front view of the medication dispensing apparatus of FIG. 1 after being manually manipulated to selectively set a dose for injection, which dose setting has shifted the actuator from the ready position shown in dashed lines in FIG. 2;

FIG. 3 is a partial front view of the medication dispensing apparatus of FIG. 2 with a front piece of the housing removed to better illustrate internal working components;

FIG. 4 is a perspective, partially exploded view of select portions of the internal workings of the apparatus of FIG. 2;

FIG. 5 is longitudinal cross-sectional view of the medication dispensing apparatus of FIG. 2, wherein FIG. 5 further differs from FIG. 2 in that the pen, as indicated by the more distal positioning of the cartridge piston, has previously been used to dispense medication from the cartridge; and

FIG. 6 is an enlarged view from FIG. 5 of the reset button of the medication dispensing apparatus.

Corresponding reference characters indicate corresponding parts throughout the several views. Although the drawings represent an embodiment of the present invention, the drawings are not necessarily to scale, and certain features may be exaggerated or omitted in some of the drawings in order to better illustrate and explain the present invention.

### DETAILED DESCRIPTION OF THE INVENTION

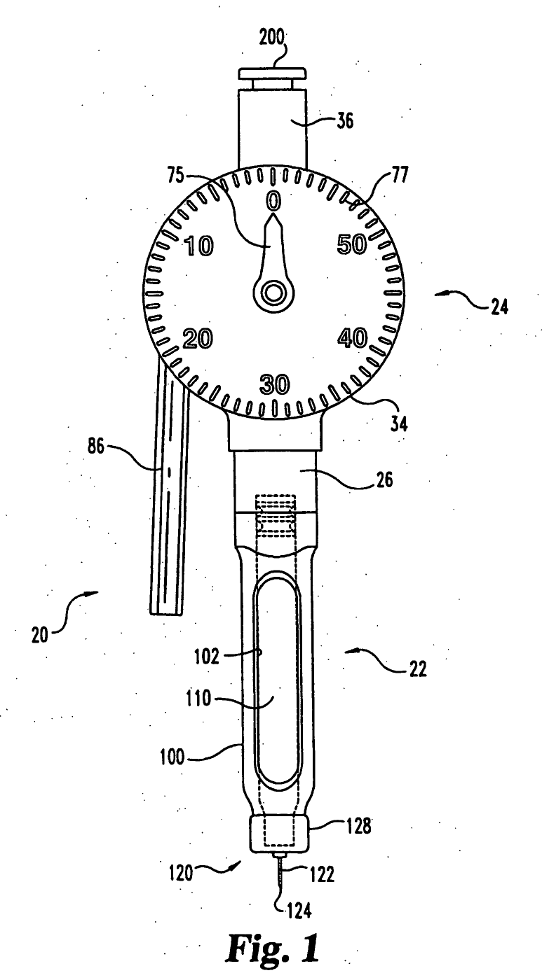

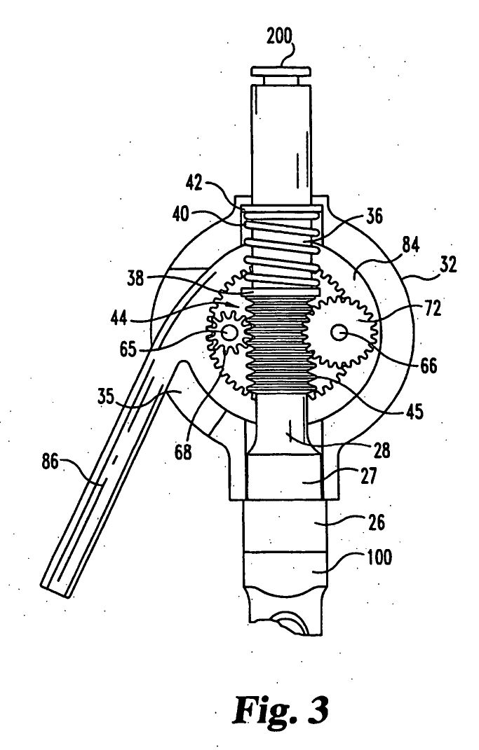

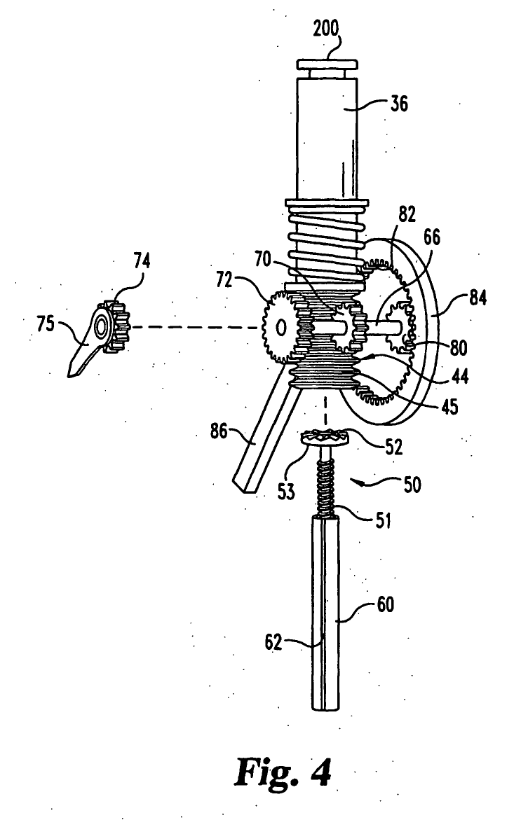

Referring now to FIGS. 1-6, there is shown a first embodiment of a medication dispensing apparatus of the present invention. Any directional references in this detailed description with respect to any of the Figures, such as right or left, upper or lower, or clockwise or counterclockwise, are intended for convenience of description, and by itself does not limit the present invention or any of its components to any particular positional or spatial orientation.

The apparatus, generally designated 20, is shown as an injection pen, which pen generally has an elongated, substantially writing instrument-like form, although other forms are within the scope of the invention. Injection pen 20 is a reusable type device and includes a distal portion 22 and a proximal portion 24. Distal portion 22 contains the medicinal fluid to be outlet at its distal end upon pen operation. The outlet end of distal portion 22 is equipped in the Figures with an injection needle. Proximal portion 24 contains the dose setting and injecting mechanism used to force a selectively set dose of medicine from the needled end.

Pen proximal portion 24 has a plastic outer housing including a tubular body section 26. A reduced diameter guide sleeve 28 is shown integrally formed with body section 26, but can be separately formed and then assembled. A housing back plate 32 is secured to a stepped down region 27 of body section 26, such as with adhesives. Back plate 32 is securely mated in a suitable manner during manufacture, such as with adhesives and alignment pins therebetween, with a housing front plate 34.

A cylindrical dosing sleeve 36 made of metal, or alternatively of plastic or other suitable material, extends upwardly from within the pen housing through a circular opening formed between the intersection of housing plates 32 and 34. Dosing sleeve 36 is shown in FIGS. 1-6 as having a shiftable reset button 200 capping its proximal end, although different capping elements, including a cap integrally formed with the dosing sleeve, may be provided. Dosing sleeve 36 includes a radially outwardly projecting flange 38 formed at a midsection of its length which remains within the pen housing during use. The upper surface of flange 38 is abutted by the distal end of a metal, helical spring 40 that is coaxially mounted on dosing sleeve 36. The proximal end of spring 40 abuts a metal washer 42 mounted around dosing sleeve 36. Washer 42 is sized large enough to engage the underside of abutting lips of housing plates 32 and 34 defining the opening through which dosing sleeve 36 projects. Spring 40 serves to bias dosing sleeve 36 downward or distally, and naturally is selected in view of the remainder of the pen design to ensure the cartridge piston is not unintentionally advanced by that spring.

The distal region of dosing sleeve 36 is made with an axially extending, cylindrical rack, generally designated 44, formed by a series of axially spaced ribs 45 that radially outwardly project from and circumferentially extend uninterrupted around the exterior of sleeve 36. Although a rack with eight ribs is shown, different rack designs or different numbers of ribs may be provided in alternate embodiments, depending on the configuration of the gear train with which rack 44 cooperates.

An internal hollow 46 of dosing sleeve 36 receives the proximal end of a drive screw 50 that axially extends therein. Within hollow 46, a radially inwardly projecting shoulder 47 of sleeve 36 has axially and distally facing teeth that mate with complementary teeth 52 axially projecting from an enlarged diameter head 53 of drive screw 50. The teeth of shoulder 47 and screw head 53 may be variously shaped, such as triangular as shown or essentially square with sloped lead-ins to ensure meshing, provided such teeth mesh to transmit rotational motion between the dosing sleeve and screw. Drive screw 50 is biased proximally by a metal helical spring 56 coaxially mounted on screw 50, which biasing forces toothed screw head 53 into toothed engagement with toothed shoulder 47. Spring 56 has a proximal end that abuts screw head 53 via an intervening bushing 58, and a distal end that abuts the top end of guide sleeve 28. In an alternate embodiment, the distal end of spring 56 may abut an additional feature provided in the inside of dosing sleeve 36 such that spring 56 travels with sleeve 36.

An external threading 51 of drive screw 50 along its shaft length engages an internal threading 61 provided along a proximal region of an axial bore-defining surface of cylindrical plunger 60. Drive screw 50 screws through the bore 63 of plunger 60, and not shown stops which inwardly project on plunger 60 into bore 63 near its distal end abut the distal end of screw 50 to thwart any manual efforts to screw plunger 60 too far upward on screw 50. Alternatively, not shown endings of threading 51 can serve as stops to thwart manual efforts to screw plunger 60 too far upward along screw 50. Drive screw 50 also preferably includes a thread stop that engages plunger threading 61 to prevent the plunger from being extended beyond its maximum length, such that the thread stop and plunger interaction prevents a user from dialing up a dose for dispensing that is greater than the medication remaining within apparatus 20. The exterior of plunger 60 includes at least one, and preferably a pair of diametrically opposed grooves 62, that longitudinally extend the entire plunger length. At least one anti-rotation prong, such as the pair of prongs 64 abstractly shown integrally formed in the interior surface of body section 26, fit within plunger grooves 62 to rotationally fix plunger 60 relative to the housing, such that only an axial sliding motion of plunger 60 within the pen housing is permitted.

A pair of axles 65 and 66, made of metal, plastic or other suitable material, are journaled to the assembled housing plates 32 and 34 on opposite sides of rack 44. A pinion 68 mounted on axle 65 has teeth that engage rack 44 only, and pinion 68 aids in centering rack 44 and thereby dosing sleeve 36 within the pen housing. As pinion 68 and axle 65 are simply provided for stability, in an alternate embodiment such parts could be eliminated and replaced with one or more stabilizing features molded into one or more of the housing elements, which features may be at multiple angular locations around the dosing sleeve to in essence provide a track in which the dosing sleeve is movable. A pinion 70 opposite pinion 68 is rotatably fixed with axle 66 and has external gear teeth that mesh with rack 44. Pinion 70, as well as the other gear elements of apparatus 20, may be made of metal, plastic or other suitable material. A pinion 72 with a larger diameter than pinion 70 is rotatably fixed with axle 66 at a location adjacent the interior face of housing front plate 34.

The gear teeth of pinion 72 mesh with teeth of a pinion 74 rotatably fixed with an axle extending through front plate 34 and on which axle is rotatably fixed a dial pointer 75. Pointer 75 is associated with a dial display 77 visible on the circular face of front plate 34, and is used to indicate a medication dose. In alternate embodiments, pen 20 can be equipped with different types of dose displays, such as an LCD display, that are operated by a dose setting and injecting assembly of the invention.

An additional pinion 80 is rotatably fixed with axle 66 and has external gear teeth that engage internal gear teeth 82 of a ring gear 84 rotatably mounted within the pen housing. An actuator in the form of an arm or lever 86 extends from ring gear 84 and projects external to the pen housing through an opening 35 provided at the interface of housing plates 32 and 34. Arm 86 is shown integrally formed with ring gear 84, but may be separately formed and fixedly assembled thereto during manufacture. When pen 20 is in a ready state, arm 86 extends generally distally from ring gear 84. Although a complete or 360° ring gear is shown, and provided the ring gear can be properly mounted within the housing, only an arcuate section of the ring gear and its teething that mate with pinion 80 is needed, which arcuate section is naturally dependent on how far the actuator arm is designed to move during the maximum dosing of injection pen 20.

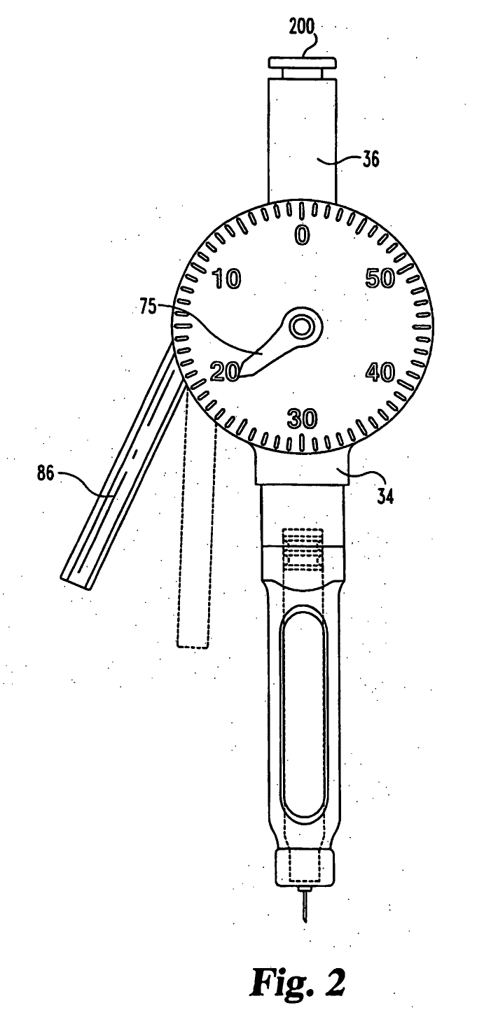

The rotation of ring gear 84 during dose setting results in arm 86 swinging or pivoting out in a clockwise direction from its position shown in dashed lines in FIG. 2 to, for example, the position shown in solid lines in FIG. 2. The swinging motion results in arm 86 being spaced farther from the pen housing in a direction transverse to the distal direction, and is therefore considered to be a movement in a direction transverse to the distal direction despite having a component of movement which is in the proximal direction. In alternate embodiments, the actuator can be other than a pivoting or swinging arm. For example, the actuator may be a four-bar type linkage by which the finger-squeezable portion of the actuator remains parallel to the housing body as it moves laterally from its ready position to any one of its dosed positions.

A clicker mechanism may also be included within the dose setting and injecting assembly, which clicker provides an audible indicator of dose size changes during dose setting, as well as an audible indicator of a unit of injection during the injection process. Such a clicker may include teeth associated with the ring gear that click over an element fixed within the housing during the ring gear rotation that occurs during dose setting and injecting, or may be any other suitable clicker mechanism known in the art.

Pinion 80 is shown separate from pinion 70, as varying the gear ratios of pinions 70 and 80 during manufacture allows different ranges of actuator motion to be provided. In the event both pinions have similar dimensions, the pinions may be integrated such that only a single gear between rack 44 and ring gear 84 is provided.

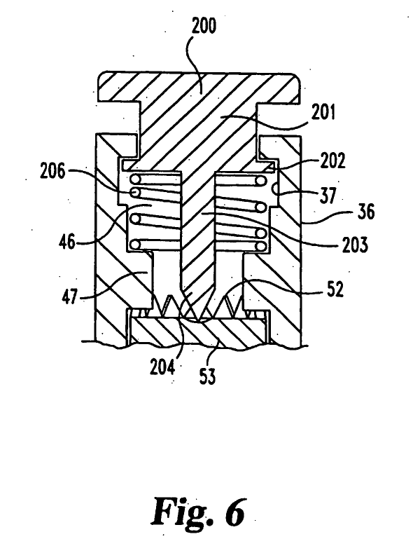

As further shown in FIG. 6, reset button 200 is provided on the proximal end of dosing sleeve 36 to facilitate plunger reset associated with the loading of a new cartridge. An opening into sleeve hollow 46 at the proximal end of dosing sleeve 36 is covered by reset button or cap 200. A circumferential rib 202 formed on a cylindrical body 201 of cap 200 radially projects within a groove 37 of dosing sleeve 36 and limits the proximal and distal motion of cap 200 relative to sleeve 36. Groove 37 has an axial length greater than the axial length of rib 202, such that cap 200 is axially shiftable a short distance from the proximal position shown in the Figures, which short distance is greater than the height of teeth 52 on screw head 53. A rod portion 203 descends from cap body 201 within hollow 46 and terminates with a conical tip 204 that extends through the central opening of toothed shoulder 47 and into engagement with the proximal face of screw head 53, which proximal face is shown with a concave surface that is abutted by the conical tip. A metal, helical spring 206 acting between shoulder 47 and body 201 biases cap 200 upwardly, which cap is captured within the dosing sleeve so as to not be removable therefrom by the force of the spring. Although the biasing force on cap 200 instead may be provided by spring 56 that biases screws head 53 upward into toothed engagement with shoulder 47, spring 206 aids in eliminating wobble of button 200.

In an alternate, not shown embodiment, the reset button can be attached to the screw head, such as with a snap fitting, so as to be axially fixed and rotatably free relative thereto. In such an embodiment, the spring 206 that biases the reset button can serve to bias the drive screw 50 proximally, and spring 56 and bushing 58 can be eliminated. Such an embodiment may facilitate reset while reducing the number of parts of the apparatus.

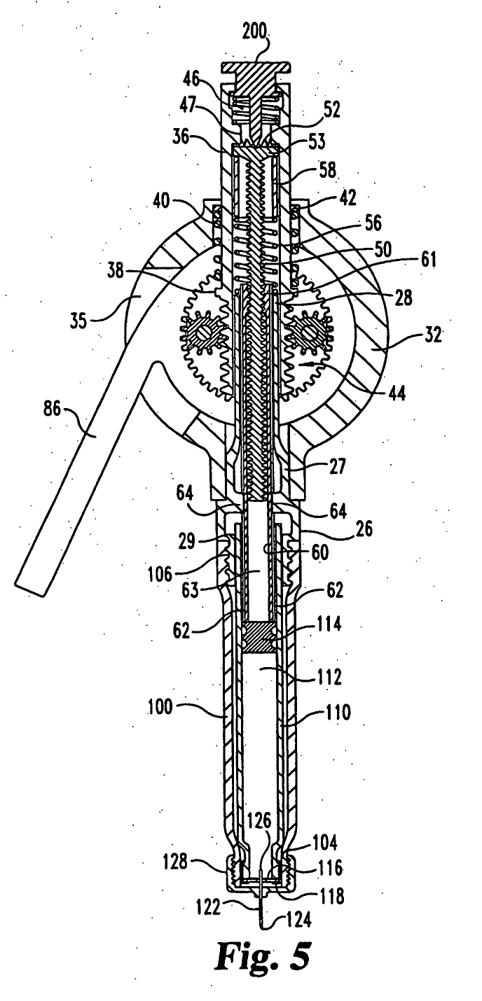

Referring again to FIGS. 1 and 5, distal portion 22 includes a reusable retainer 100 with a replaceable medication cartridge 110 removably held therein. Windows 102 in retainer 100 allow the contents of cartridge 110 to be seen to permit a user to estimate medicine remaining. The open, stepped-down distal end of retainer 100 is provided with external threading 104, or other suitable connection means, to releasably connect a pen-needle assembly, generally designated 120. Internal threads 106 connect with external threading 29 on a stepped down region of body section 26 to removably mount the cartridge retainer 100 to the injection pen housing.

Pen-needle assembly 120 is of known design and includes a double-ended needle cannula or injection needle 122 having a distal tip 124 at one end and a proximal point 126 at the other. Injection needle 122 is mounted in a tubular hub 128 that is internally threaded to cooperate with the shown retainer design so as to be screwable onto and off of threading 104 of the retainer distal end. Although the needle assembly is shown as having a single injection needle, needle assemblies which may be used with pen 20 may be of various types known in the art, including, but not limited to, assemblies with one or more shortened injection needles, including microneedle arrays.

Cartridge 110 is of a conventional design and defines a medicine-filled reservoir 112 that is closed at its proximal end by a piston 114 that is axially slidably and sealably engaged with the cartridge interior wall to hold the fluid medication within reservoir 112. The distal, outlet end of cartridge reservoir 112 is sealed by a septum 116 held by a cap 118 that is secured to a stepped-down diameter neck portion of the cartridge. When pen-needle assembly 120 is mounted on threading 104, the proximal point 126 of injection needle 122 penetrates cartridge septum 116 to provide a fluid flow outlet by which medicine within cartridge reservoir 112 can be dispensed from the needle tip during operations of injection pen 20.

The fluid medicine container shown and described above is illustrative and not intended to be limiting as other constructions may be employed within the scope of the invention. For example, rather than the shown container in which a distinct cartridge is held within a removable retainer, in another fluid container embodiment, the cartridge could be constructed to be sufficiently durable and adapted to secure directly to pen proximal portion 24 -without any protective retainer therearound, and with the pen-needle assembly directly mountable to the cartridge, or the fluid container could be a combination cartridge/retainer which is disposable as a unit. Still further, in the case of a disposable injection pen, the cartridge-holding retainer could be fixedly mounted or secured, via adhesives, ultrasonic welding or in another suitable manner, to a previously subassembled pen proximal portion 24 when injector pen 20 is assembled by the manufacturer.

The structure of injection pen 20 will be further understood in view of the following explanation of its operation. Initially, a user requiring a dose of medication will locate pen 20, which pen is typically in the ready arrangement shown in FIG. 1, which is the arrangement in which the pen remained after its previous use, or in which the pen is provided to a user for its first use.

Pen 20 should first be primed, which priming step will not be further described as it involves operating the pen with a small set dose and in the same way as described further below, except that in the priming process, the pen is pointed upward into the air during operation as is conventional to eliminate any air from the needle and cartridge.

After priming, pen 20 is ready to be used for injection. Pen 20 is first manipulated by the user to select the dose desired to be administered by operation of the pen. Specifically, while holding pen 20 without preventing motion of actuator arm 86, dosing sleeve 36 is rotated relative to the pen housing in order to selectively set the dose to be dispensed. During this rotation, drive screw 50, due to its toothed engagement with the dosing sleeve shoulder 47, rotates with dosing sleeve 36. The threaded engagement between the rotatably fixed plunger 60 and drive screw 50 causes dosing sleeve 36 and screw 50 to screw upward from plunger 60 such that dosing sleeve 36 projects from the housing farther proximally than before. This axial motion of dosing sleeve 36 and drive screw 50, while plunger 60 remains axially stationary, is due to the resistance to dosing sleeve and drive screw motion being less than frictional forces required to be overcome to advance plunger 60 and cartridge piston 114. An additional element acting between plunger 60 and the pen housing to add additional frictional resistance to such axial motion of plunger 60 within the pen housing may also be employed. Dosing sleeve 36 is prevented from being rotated to set a dose larger than the medication remaining in the cartridge due to the interaction of the thread stop of the drive screw 50 with plunger 60 as described above.

As dosing sleeve 36 moves proximally during dose setting against a returning force provided by spring 40, rack 44 similarly moves proximally. During this longitudinal motion of rack 44, its engagement with pinion 70 rotates that pinion so as to produce a rotation of pinions 80 and 72. The rotation of pinion 72 results in a rotation of pinion 74 and dial pointer 75, which is appropriately calibrated to point to the amount of medication that the pen is then set to deliver. The rotation of pinion 80 rotates ring gear 84, which ring gear rotation causes arm 86 to swing out clockwise from the ready position to another position more laterally spaced from the housing. The arc through which actuator arm 86 swings during dose setting is directly proportional to the dose being set. Thus, the maximum dose that the pen can be set to deliver in a single operation, such as sixty units for the shown device, results in the swing arm moving out twice as far as if the injection pen 20 was set to deliver thirty units upon its operation. Actuator arm 86 therefore can be shifted during selective dose setting to any of a plurality of possible dosed positions, each dosed position corresponding to a particular set dose. At this point, pen 20 is arranged in the ready-to-inject state shown in FIG. 2, in which the selectively set dose is approximately twenty-one units.

To actually inject the medicine, after pen 20 is manipulated so injection needle distal tip 124 properly penetrates, for example, a user's skin, actuator arm 86 is squeezed inward toward the pen housing, typically with one or more fingers of the hand in which the pen 20 is being held. As arm 86 is squeezed and thereby swings back toward the ready position, ring gear 84 is rotated to force a counter clockwise rotation of pinion 80, which simultaneously results in a counter clockwise rotation of pinion 70 which forces rack 44 distally. This distal motion of rack 44 causes a corresponding axial motion of drive screw 50 and plunger 60, and the distal end of plunger 60 forces piston 114 distally to decrease the reservoir volume so as to force medication from needle 122 into the user. During this actuator arm motion, dial pointer 75 rotates down to indicate the amount of medication still to be injected during further actuator arm motion. The injection is completed when actuator 86 has been fully laterally shifted back to its home position, at which time pen 20 is once again arranged in the ready or ready-to-be set state shown in FIG. 1.

Pen 20 can continue to be used to deliver medicine until it cannot be manipulated to set a desired dose, due to such desired dose being greater than the amount of medication remaining in the cartridge. The user may then remove retainer 100, and replace the spent cartridge with a replacement cartridge 110. Then, a force sufficient to overcome the spring forces is manually applied to plunge cap 200 distally relative to dosing sleeve 36, which plunging moves conical tip 204 downward a sufficient distance to cause a disengagement of the toothed connection between shoulder 47 and drive screw head 53. When an upward force is then applied on plunger 60 while cap 200 is plunged, which force can be manually applied or provided by the cartridge piston as a cartridge-holding retainer is mounted to the pen proximal portion, the plunger 60 is moved upward which spins the drive screw 50 on conical tip 204. Thus, the dosing sleeve 36 is not forced to rotate during plunger reset. After such plunger retraction, the retainer 100 with replacement cartridge 110 is installed to the pen proximal portion 24, and the pen is ready for use.

While this invention has been shown and described as having preferred designs, the present invention may be modified within the spirit and scope of this disclosure. For example, rather than the shown ring gear and rack and pinion engagement, the actuator arm may eliminate the ring gear and be directly connected to a pinion, which pinion may directly engage a rack or be provided with gearing therebetween. Still further, in an alternate embodiment, the reset button 200 is eliminated, and the dosing sleeve 36 and screw 50 of the embodiment of FIGS. 1-6 are integrally formed or fixedly connected together. In such an embodiment, in which spring 56 and bushing 58 are also eliminated, reset of the plunger in anticipation of loading of a new cartridge may be accomplished by manually rotating the dosing sleeve with integral or fixedly connected screw a sufficient number of times to retract the plunger. This application is therefore intended to cover any variations, uses or adaptations of the invention using its general principles. Further, this application is intended to cover such departures from the present disclosure as come within known or customary practice in the art to which this invention pertains.

We claim:

1. A medication dispensing apparatus comprising:
a housing;
a drive member movable within said housing;
a fluid container defining a medicine-filled reservoir with a movable piston at one end and an outlet at the other end, said piston engagable by said drive member to be advanced toward said outlet in a distal direction when said drive member is moved; and
a dosing and injecting assembly for selectively shifting said drive member, said dosing and injecting assembly including an actuator and a dosing member, said dosing member operable to selectively set a dose to be delivered, said actuator movable relative to said housing in a direction transverse to said distal direction between a ready position and any one of a plurality of dosed positions, wherein said actuator, responsive to said dosing member being operated to selectively set a particular dose to be delivered, moves from said ready position to a particular dosed position that corresponds to the particular dose selectively set, and wherein said drive member, responsive to said actuator being manually shifted from said particular dosed position back to said ready position, moves to shift said piston distally to cause said set dose to be delivered through said outlet.

2. The medication dispensing apparatus of claim 1 wherein a distance said actuator moves in the direction transverse to said distal direction during movement from said ready position is proportional to a size of the selected dose.

3. The medication dispensing apparatus of claim 2 wherein said actuator comprises a pivoting element that swings away from said housing when moved from said ready position to any one of said plurality of dosed positions, and wherein the angular size of the arc through which said pivoting element swings during movement from said ready position is proportional to a size of the selected dose, whereby the position of said pivoting element serves as a visible indicator of relative dose size.

4. The medication dispensing apparatus of claim 1 wherein said dosing and injecting assembly includes a rack and pinion gearing between said actuator and said dosing member.

5. The medication dispensing apparatus of claim 4 wherein a portion of said dosing member within said housing comprises said rack.

6. The medication dispensing apparatus of claim 4 wherein said actuator comprises at least a portion of a ring gear in engagement with a pinion rotatable with said pinion of said rack and pinion gearing.

7. The medication dispensing apparatus of claim 6 wherein said pinion in engagement with said ring gear, and said pinion of said rack and pinion gearing, are arranged coaxially.

8. The medication dispensing apparatus of claim 1 wherein said dosing member is manually rotatable to selectively set the dose, wherein rotation of said dosing member to increase the dose setting causes said dosing member to move in a direction opposite the distal direction from a retracted position to one of a plurality of extended positions, said dosing member, when disposed in any one of said plurality of extended positions, projecting farther out from said housing than when disposed in said retracted position.

9. The medication dispensing apparatus of claim 8 further comprising means for biasing said dosing member from any of said plurality of extended positions toward said retracted position.

10. The medication dispensing apparatus of claim 8 wherein said dosing member, responsive to said actuator being manually shifted from any one of said plurality of dosed positions to said ready position, axially moves without rotating from any one of said plurality of extended positions back to said retracted position.

11. The medication dispensing apparatus of claim 1 wherein said dosing and injecting assembly includes a drive screw rotatable with said dosing member and threadedly engaged with said drive member, wherein said drive member is rotatably fixed within said housing such that rotation of said drive screw produces an axial motion of said drive member relative to said drive screw.

12. The medication dispensing apparatus of claim 11 further comprising means for permitting said drive screw to rotate relative to said dosing member for drive member resetting.
